SEQUENCE LISTING (5 sequences)

SEQ ID NO: 1 - DNA, 1410 bp, Artificial Sequence
  nucleic acid sequence of VEGF-Trap
tccccgcggggagccaccatggagacagacacactcctgctatgggtactgctgctctgggttccaggatccactggtagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgattaagaaaaaatacgggtagaaggtttaaaccc

SEQ ID NO: 2 - DNA, 18931 bp, Artificial Sequence
  genome sequence of the recombinant Newcastle disease virus rNDV-VEGF-Trap
agttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataagcatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaagcggccgctaatacgactcactatagggaccaaacagagaatccgtaagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcctcagccccactgaatgatcgcgtaaccgtaattaatctagctacatttaagattaagaaaaaatacgggtagaatccccgcggggagccaccatggagacagacacactcctgctatgggtactgctgctctgggttccaggatccactggtagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgattaagaaaaaatacgggtagaaggtttaaacccttggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggccccaaatcttctaccaatgtcccagcacctctgatgctgaccgtcaggattgcgctggcactgagctgtgtccgtctgacaaattctctcgatggaaggcctcttgcagctgcagggattgtagtaacgggagacaaagcagtcaacatatacacctcatctcagacagggtcaataatagtcaagttactcccaaatatgcctaaggataaagaggcgtgtgcaaaagccccgttggaggcatacaacaggacactgactactttgctcaccccccttggtgattctatccgcaggatacaagagtctgcgactacgtccggaggaaggaggcagagacgctttataggtgccattatcggcagtgtagctcttggggttgccacagatgcccagataacagcagcctcagctctgatacaagccaaccagaatgctgccaacatcctccggcttaaagagagcattgctgcaactaatgaagctgtacatgaagtcactgacggattatcgcaactagcagtggcagttgggaagatgcagcagtttgttaatgaccagtttaataacacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtggtaatatggattatttgttgactaagttaggtgttgggaacaaccaactcagctcattaatcggtagcggcttgatcaccggtaaccctattctgtacgattcacagactcaactcttaggtatacaggtaactttaccctcagtcggtaacctaaataatatgcgtgctacctacttggagaccttgtctgtaagcacaaccaagggatttgcctcagcacttgtcccaaaagtggtgacacaggtcgggtctgtgatagaggaacttgacacctcatactgtgtagagaccgatttggatttatattgtacaagaatagtgacattccctatgtctcctggtatttattcctgtctgagcggtaatacatcagcttgcatgtattcaaagactgaaggtgcacttactacgccatatatgactatcaagggctcagttattgccaattgcaagatgacaacatgcagatgtgcagaccctccgggtatcatatcgcaaaattatggagaagctgtgtctctaatagataggcactcatgcaatgtcttatccttagacgggataactttgaggctcagtggagaatttgacgtaacttatcaaaagaatatctcaatattagattctcaggtaatagtgacaggcaatctcgatatctcaactgaacttgggaatgtcaacaactcgataagtaatgctttggataagttagaggaaagcaatagcaaacttgacaaagtcaatgtcaagctgaccggcacgtctgctctcattacctatatagttttaactatcatatctcttgtttgtggtatacttagcctggttctagcatgctatctgatgtataagcaaaaggcgcaacaaaagaccttattatggcttgggaataataccctaaatcagatgagggccactacaagaatctgaacacagatgaggaacgaaggtttccctaatagtaatttgtgtgaaagttctggtagtctgtcagttaagaaaaaatacgggtagaaggttaaccggcccgcggcgacgcgtggcctgagaggccttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggtaaataatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaataacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagtcccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtttggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatcggccgatccggctgctaacaaagcccgaaaggaagctgagttggctgctgccaccgctgagcaataactagcataaccccttggggcctctaaacgggtcttgaggggttttttgctgaaaggaggaactatatccggatggccgccaccggtgggccttgcagcacatccccccttcgccag

SEQ ID NO: 3 - DNA, 23 bp, Artificial Sequence
  upstream primer
tcaagcgccttgctctaaatggc

SEQ ID NO: 4 - DNA, 25 bp, Artificial Sequence
  downstream primer
gggcagaatcaaagtacagcccaat

SEQ ID NO: 5 - DNA, 18931 bp, Artificial Sequence
  genome sequence of the recombinant Newcastle disease virus rClone30-Anh-(F)-VEGF-Trap
agttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataagcatgagtgataacactgcggccaacttacttctgacaacgatcggaggaccgaaggagctaaccgctttttttcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacgggcagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccaggggggaacgcctggtatctttatagtcctgtcgggtttcgccacctctgacttgagcgtcgatttttgtgatgctcgtcaggggggccgagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttacctcactcattaggcaccccaggctttacactttatgcttccggctcctatgttgtgtggaattgtgagcggataacaatttcacacaggaaacagctatgaccatgattacgccaagctcggaagcggccgctaatacgactcactatagggaccaaacagagaatccgtaagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactccgcacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctccaagcggcaatcctctctcgcttcctcagccccactgaatgatcgcgtaaccgtaattaatctagctacatttaagattaagaaaaaatacgggtagaatccccgcggggagccaccatggagacagacacactcctgctatgggtactgctgctctgggttccaggatccactggtagtgatacaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgcgaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgattaagaaaaaatacgggtagaaggtttaaacccttggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccactgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagaaggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggccccaaaccccccaccggaaccccagcgcctctggtgctgatcgcccggaccgcgctggcgttgggctgtgtctgtccggcgggctctcttgacggcagacctcttgcagctgcagggattgtggtaacgagagataaagcagtcaatatatacacttcatctcaaacggggtcaatcatagtcaagttactcccaaatatgcccaaagacaaggaggcgtgcgcaaaagccccattagaggcgtacaatagaacactgaccactttactcactcctcttggcgactccatccgcaggatacaagggtctgcaactacatctagaggaaggagacagaaacgttttgtaggtgctatcattggcagtatagctcttggggttgcgacagctgcacaagtaacagcagctgcagctctgatacaagccaaccagaacgctgccaacatcctccggcttaaggagagcattgctgcaaccaatgaagctgtgcacgaggtcactgacggattatcacaactagcgatggcgattgggaagatgcagcagtttgttaatgaccagtttaataatacggcgcgagaattggactgcatcaaaattacacaacaggttggtgtcgaactcaatttgtatctaactgaactgactacagtattcgggccacaaatcacttcccctgctttaactcagctaactatccaggcactttataatttagctggtggcaatatgaattacttattgactaagttaggtgtagggaacaatcaacttagctcattaattagtagtggcctgatcactggcaaccccattttatatgactcacagacccaactcttaggcatacagataaatgtaccctcagtcgggagcctaaataatatgcgtgccacctacttggagaccttatccgtaagcacaaccagggggttcgcctcagcacttgtcccgaaagttgtgacgcaagttggttctgtgatagaagaacttgacacctcatattgtatagaatctaatctggatttatattgtacaaggatagtgacattccccatgtctcccggcatttattcctgtctgagcggtaatacgtcagcttgtatgtattcaaagactgagggtgcactcactacaccatacatagctctcaagggctcagttattgctaattgcaagatgattacatgtagatgtgcagaccccccaggtatcatatcgcaaaattacggagaagctgtgtccctaatagataaacattcatgtaatgtcttatccctagacggaataaccctgaggctcagtggggaatttgatgcgacctatcaaaagaacatcttaatactagattcccaggtcatcgtgacaggcaatctcgatatatcaactgaacttgggaatgtcaacaactcgataagcagtgctctggacaaattagcggaaagtaacagcaagttaaacaaagtcaatgtcaacctaactagcacatctgctctcattacttatattgttctagctgtcatatctcttgttttcggcgtaattagcctgattctagcgtgctgcttgatgtataaacaaaaagcacaacaaaagaccttactatggcttgggaacaataccctcgatcagatgagagccaccacaaaaacatgaacacagatgaggaacgaaggtttccctaatagtaatttgtgtgaaagttctggtagtctgtcagttaagaaaaaatacgggtagaaggttaaccggcccgcggcgacgcgtggcctgagaggccttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggtaaataatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaataacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctagacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagtcccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagtgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggtttggtggccggcatggtcccagcctcctcgctggcgccggctgggcaacattccgaggggaccgtcccctcggtaatggcgaatgggacgcggccgatccggctgctaacaaagcccgaaaggaagctgagttggctgctgccaccgctgagcaataactagcataaccccttggggcctctaaacgggtcttgaggggttttttgctgaaaggaggaactatatccggatcggccgatccggctgctaacaaagcccgaaaggaagctgagttggctgctgccaccgctgagcaataactagcataaccccttggggcctctaaacgggtcttgaggggttttttgctgaaaggaggaactatatccggatggccgccaccggtgggccttgcagcacatccccccttcgccag

(19) United States
(12) Patent Application Publication
(10) Pub. No.: US 2024/0124852 A1
(43) Pub. Date: Apr. 18, 2024

(54) RECOMBINANT NEWCASTLE DISEASE VIRUS RNDV-VEGF-TRAP, GENOME THEREOF, PREPARATION METHOD THEREFOR, AND USE THEREOF

(71) Applicant: Jiangsu Kanionreal Biomedical Technology Co., Ltd., Lianyungang (CN)

(72) Inventors: Wei Xiao, Lianyungang (CN); Deshan Li, Lianyungang (CN); Tianyan Liu, Lianyungang (CN); Zhenzhong Wang, Lianyungang (CN); Yukai Cao, Lianyungang (CN); Zhihang Liu, Lianyungang (CN); Dan Yu, Lianyungang (CN)

(21) Appl. No.: 18/554,972

(22) PCT Filed: Apr. 13, 2022
(86) PCT No.: PCT/CN2022/086599

§ 371 Date: Oct. 11, 2023

(30) Foreign Application Priority Data
Apr. 13, 2021 (CN) 202110393844.7

(51) Int. Cl. C12N 7/00; A61K 35/768; A61P 35/00; C07K 14/005

(52) U.S. Cl. CPC C12N 7/00; A61K 35/768; A61P 35/00; C07K 14/005; A61K 38/00; C12N 2760/18121; C12N 2760/18122; C12N 2760/18132

(57) ABSTRACT

The present application discloses a recombinant Newcastle disease virus genome, a recombinant Newcastle disease virus rNDV-VEGF-Trap containing the genome and a preparation method therefor, a DNA molecule encoding the recombinant Newcastle disease virus genome, and a use of the genome and the recombinant Newcastle disease virus in the preparation of a drug for treating cancer. The recombinant Newcastle disease virus provided by the present application relates to inserting a coding gene of VEGF-Trap into the genome of the recombinant Newcastle disease virus, such that the recombinant Newcastle disease virus obtained therefrom is replicated with a strong replication capability, thereby killing host cancer cells; moreover, the recombinant Newcastle disease virus has reliable safety for non-cancer cells, and shows improved anti-tumor effect and oncolytic efficiency.

### FIELD OF THE INVENTION

The present application belongs to the field of oncolytic virus for cancer treatment, and particularly relates to a recombinant Newcastle disease virus genome, a recombinant Newcastle disease virus comprising the genome and a preparation method therefor, a DNA molecule encoding the recombinant Newcastle disease virus genome, and use thereof.

### BACKGROUND OF THE INVENTION

Cancer is a disease caused by the loss of normal regulation and excessive proliferation of body cells. At present, it has become the first killer affecting health. China is an area with high incidence of cancer, especially lung cancer, gastric cancer, liver cancer and rectal cancer. According to statistics, in 2016 alone, 4.8 million new cases of various cancer patients emerged in China, and 2.3 million patients died from various cancers. With the progress of technology, various new treatment means, especially biopharmaceutical therapy, are continuously put into clinical use. However, the needs for drug safety, effectiveness and quality of life of patients are far from being met. The development of new drugs or treatment means is imperative.

In 1991, Martuza et al. published an article in Science, demonstrating that transgenic herpes simplex virus has a certain effect in the treatment of glioblastoma. Since then, the development of oncolytic viruses to treat cancer has attracted increasing attention. The principle of oncolytic virus treatment of cancer is to genetically modify some naturally occurring viruses with weak pathogenicity, allowing them to selectively infect tumor cells, replicate extensively within cells, and ultimately destroy tumor cells. At the same time, it can also stimulate immune responses, and attract immune cells to continue killing remaining cancer cells or kill migrated cancer cells through immune responses. In recent decades, researches on oncolytic viruses have made tremendous progress. Newcastle disease virus (NDV), herpes simplex virus 1 (HSV-1), reovirus, and oncolytic adenovirus have been successively used to develop oncolytic viruses, but their clinical manifestations are far below expectations. For example, in 2005, the CFDA approved the oncolytic adenovirus product H101 for marketing, but its therapeutic effect was not ideal.

NDV is an avian paramyxovirus with a negative-sense single-stranded RNA genome, which has always been a promising method for cancer treatment. However, the effect of NDV as a single drug treatment is limited. In one aspect, the antiviral immune response in human body can clear the virus. In another aspect, the human body can produce neutralizing antibodies to resist the viruses and exert the effects. In order to improve the therapeutic efficiency of NDV in cancers, these viruses are subsequently used to deliver genes with anti-tumor activity to further enhance their activity. Such genes include genes encoding cytokines or their receptors, immune checkpoint molecules, tumor suppressor proteins, or immune stimulatory proteins.

Numerous studies on recombinant Newcastle disease viruses, into the genomes of which genes encoding cytokines or their receptors are integrated, have previously been conducted in the field, but no clinically beneficial progress has been made. For example, Pascal Buijs et al. (Recombinant Immunomodulating Lentogenic or Mesogenic Oncolytic Newcastle Disease Virus for Treatment of Pancreatic Adenocarcinoma, Viruses 2015, 7, 2980-2998) studied the recombinant Newcastle disease virus expressing interferon or interferon antagonist protein.

Researches show that angiogenesis is an important target for tumor treatment. Angiogenesis is the process of generating new blood vessels from existing endothelial cells to provide sufficient oxygen and nutrients to various organs, which is crucial for tumor growth and metastasis. Anti-angiogenic therapy is one of the important methods for cancer treatment. During the tumor neovascularization, pro-angiogenic factors supporting tumor growth mainly involve vascular endothelial growth factor (VEGF), platelet-derived growth factor (PDGF), epidermal growth factor (EGF), angiogenin, and transforming growth factor β(TGF-β). These factors activate downstream signaling pathways by binding to corresponding receptors, thereby regulating the formation of new blood vessels in tumors.

Most anti-angiogenic drugs target pro-angiogenic factors and their receptors, or key molecules in downstream signaling pathways, thereby inhibiting tumor growth and metastasis via blocking the nutrient supply to tumors. The anti-angiogenic drugs currently approved by the FDA mainly include macromolecular monoclonal antibodies and small molecule targeted inhibitors. Among them, anti-angiogenic factors mainly include thrombospondin 1 (TSP-1), angiostain, endostain, and interferon-α (IFN-α), etc., as well as VEGF blockers/antagonists such as VEGF-Trap (obtained by fusing the Ig domain of VEGFR with the constant region of IgG molecules). These inhibitors can directly inhibit the proliferation and migration activity of vascular endothelial cells, thereby inhibiting angiogenesis and blocking tumor growth and metastasis, and can show beneficial effects in cancer treatment. However, anti-angiogenic drugs have significant therapeutic side effects.

In view of the problems such as the limited therapeutic effect of NDV as a single drug, low response rate and low tumor inhibition rate in the prior art that limit the clinical application of oncolytic viruses, further research is still needed on recombinant NDV expressing exogenous proteins with anti-tumor activity that can improve anti-tumor efficacy and reduce side effects.

### SUMMARY OF THE INVENTION

The inventors of the present application provide a corresponding recombinant oncolytic virus by integrating the encoding genes of Angiostatin and VEGF-Trap into specific positions of the Newcastle disease virus genome. After being verified via pharmacodynamic tests, it is found that the anti-tumor effect of the recombinant oncolytic virus rNDV-VEGF-Trap is significantly higher than that of the rNDV group and rNDV-Angiostatin group. It can replicate in cancer cells with strong replication ability to kill host cancer cells, while having reliable safety for non-cancer cells, thus solving the above technical problem.

In one aspect, the present application provides a recombinant Newcastle disease virus genome, wherein the genome comprises a gene encoding VEGF-Trap located between P gene and M gene of the Newcastle disease virus genome.

In another aspect, the present application provides a recombinant Newcastle disease virus, wherein the virus comprises the above-mentioned recombinant Newcastle disease virus genome.

In still another aspect, the present application provides a DNA molecule encoding the above-mentioned recombinant Newcastle disease virus genome.

In still another aspect, the present application provides a pharmaceutical composition, wherein the pharmaceutical composition comprises the above-mentioned recombinant Newcastle disease virus genome, recombinant Newcastle disease virus and/or DNA molecule.

In still another aspect, the present application provides a method for preparing the above-mentioned recombinant Newcastle disease virus, wherein the method comprises:
(1) performing enzymatic cleavage on a cloning vector comprising DNA sequence of a VEGF-Trap encoding gene and a NDV viral vector, respectively, and conducting a ligation between the DNA sequence of the VEGF-Trap encoding gene and the NDV viral vector resulted from the enzymatic cleavage, so as to obtain a recombinant Newcastle disease virus plasmid;
(2) transfecting the recombinant Newcastle disease virus plasmid into cells and culturing the transfected cells to obtain the recombinant Newcastle disease virus.

In still another aspect, the present application provides use of the above-mentioned recombinant Newcastle disease virus genome, recombinant Newcastle disease virus, DNA molecule and/or pharmaceutical composition in the preparation of a medicament for treating or improving cancer. Alternatively, the present application provides the above-mentioned recombinant Newcastle disease virus genome, recombinant Newcastle disease virus, DNA molecule and/or pharmaceutical composition for use in treating or improving cancer. Alternatively, the present application provides use of the above-mentioned recombinant Newcastle disease virus genome, recombinant Newcastle disease virus, DNA molecule and/or pharmaceutical composition for treating or improving cancer. Alternatively, the present application provides a method of treating or improving cancer, comprising administering to a subject in need thereof the above-mentioned recombinant Newcastle disease virus genome, recombinant Newcastle disease virus, DNA molecule and/or pharmaceutical composition.

By integrating the VEGF-Trap encoding gene into a specific position of the Newcastle disease virus genome, the anti-tumor effect and oncolytic efficiency of the obtained recombinant oncolytic virus can be significantly improved.

### DESCRIPTION OF THE DRAWINGS

In order to more clearly illustrate the exemplary technical solutions of the present application, the accompanying drawings will be briefly introduced below. It should be understood that the following drawings only illustrate the exemplary technical solutions of the present application, and thus should not be regarded as limiting the protection scope.

FIG. 1 shows the Western Blot detection results of the allantoic fluid in Example 1, wherein the recombinant Newcastle disease virus rNDV-VEGF-Trap prepared in Example 1 can stably express the exogenous gene VEGF-Trap.

FIG. 2 shows the proliferation curves of each recombinant Newcastle disease virus and the parental virus inoculated into DF-1 cells.

FIG. 3 shows the tumor growth curves in mice of the negative control group and each recombinant Newcastle disease virus treatment group and the parental virus treatment group.

FIG. 4 shows the tumor inhibition results of mice in the negative control group and each recombinant Newcastle disease virus treatment group and the parental virus treatment group.

FIG. 5 is a picture showing tumors in mice of the negative control group and each of the recombinant Newcastle disease virus treatment group and the parental virus treatment group.

FIG. 6 shows the HE staining results of the negative control group and each recombinant Newcastle disease virus treatment group and the parental virus treatment group, wherein the tumor tissue structure of the mice in the negative control group is dense, with intact cell morphology and vigorous growth; the tumor lesions of the mice in the rNDV group are disintegrated and the tumor cell structure is relatively loose; the tumor structure of the mice in the rNDV-Angiostatin group is not significantly different from that of the rNDV group, while the tumor tissue lesions of the mice in the rNDV-VEGF-Trap group are extensively disintegrated, the tumor cell structure is very loose, immune cells infiltrate in multiple locations, and the tumor cells are scattered individually.

FIG. 7 shows the immunohistochemical staining results, in which the expression of CD34 in the mice of the negative control group is abundant, the rNDV group is similar to the negative control group, while the expression of CD34 in the mice of the rNDV-VEGF-Trap group is significantly reduced.

FIG. 8 shows the inhibition of tumor growth in mouse liver cancer model by rClone30-Anh-(F) treatment group, rClone30-Anh-(F)-Angiostatin treatment group and rClone30-Anh-(F)-VEGF-Trap treatment group.

FIG. 9 shows the genome sequence of the recombinant Newcastle disease virus rNDV-VEGF-Trap prepared in Example 1.

FIG. 10 shows the genome sequence of the recombinant Newcastle disease virus rClone30-Anh-(F)-VEGF-Trap prepared in Example 5.

### EMBODIMENTS OF THE INVENTION

Exemplary embodiments of the present application are described below, but the protection scope of the present application is not limited hereto. Unless otherwise defined, the technical and scientific terms used herein have the same meanings as commonly understood by the person of ordinary skill in the art to which this disclosure belongs, which can be seen in, for example, Singleton et al., Dictionary of Microbiology and Molecular Biology 2nd ed., J. Wiley & Sons (New York, NY 1994); Sambrook et al., Molecular Cloning, A Laboratory Manual, Cold Springs Harbor Press (Cold Springs Harbor, N Y 1989).

Unless otherwise specified, the term “treatment” herein means curing, reducing, alleviating, slowing down, palliating or ameliorating a disease or related symptoms in a statistically significant manner, or preventing, delaying, stopping, discontinuing or halting the onset or further development of a disease or related symptoms.

Unless otherwise specified, all the numbers used herein to express amounts of ingredients, measured values, or reaction conditions should be understood as being modified in all cases by the term “about” to indicate possible measurement errors. For example, when being connected to a percentage, the term “about” can represent a variation within a range of ±1%, such as ±0.5%, of the object value to which it limits.

Unless otherwise specified, a singular term herein encompasses a plural referent and vice versa. Likewise, unless clearly indicated otherwise in the context thereof, the term “or” intends to include “and” and vice versa.

Unless otherwise specified, the terms “comprise, comprises and comprising” or their equivalents (e.g., contain, containing, include, including) herein are open-ended expressions and should be understood as “include but not limited to”, which means that in addition to the listed elements, components and steps, other unspecified elements, components and steps may also be covered.

The identity percentage (degree of homology) between sequences herein can be determined by aligning the two sequences using, for example, a freely available computer program commonly used for this purpose on the World Wide Web, such as BLASTp or BLASTn with default settings.

Newcastle disease virus (NDV) belongs to the order Mononegavirales, family Paramyxoviridae, and has an envelope; the nucleocapsid is located within the envelope and contains the RNA genome and nucleocapsid protein. The genome length of the classic Newcastle disease virus is about 15-16kb, comprising NP gene, P gene, M gene, F gene, HN gene and L gene in the direction of 3′ end to 5′ end, which are used to encode the following 6 main proteins: Nucleocapsid Protein (NP), Phosphate Protein (P), Matrix Protein (M), Fusion Protein (F), Haemagglutinin Neuraminidase Protein (HN), and Large Protein (L).

In one embodiment, the present application relates to a recombinant Newcastle disease virus genome, wherein the genome comprises a VEGF-Trap encoding gene, and the VEGF-Trap encoding gene is located between P gene and M gene of the Newcastle disease virus genome.

In some preferred embodiments, the VEGF-Trap encoding gene may be in the form of DNA or RNA.

In some preferred embodiments, the VEGF-Trap encoding gene has the sequence set forth in SEQ ID NO. 1 or a sequence having at least 80% (such as 80%, 85%, 90%, 91%, 92%, 93%, 94%, 95%, 96%, 97%, 98% or 99%) identity thereto.

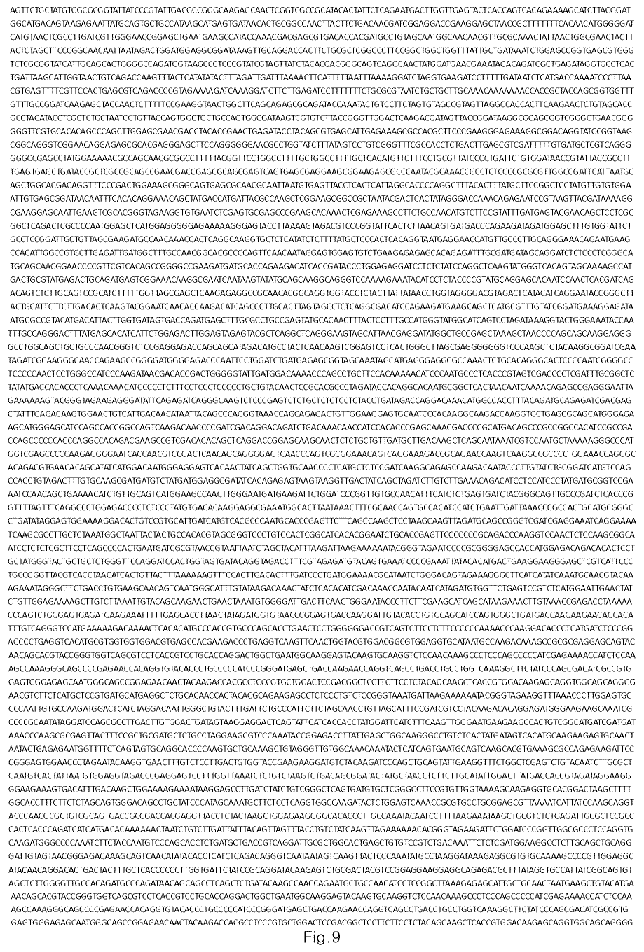

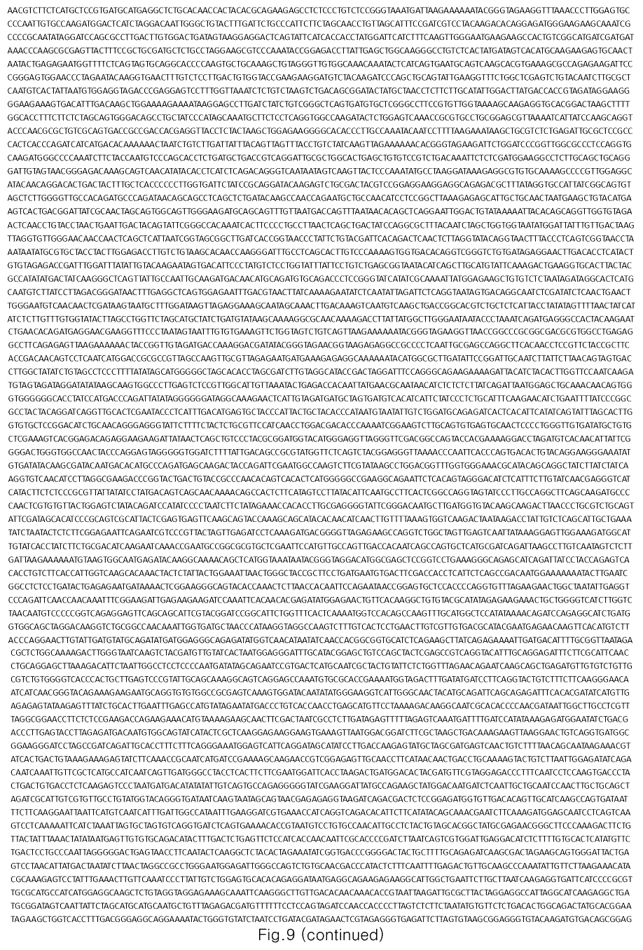

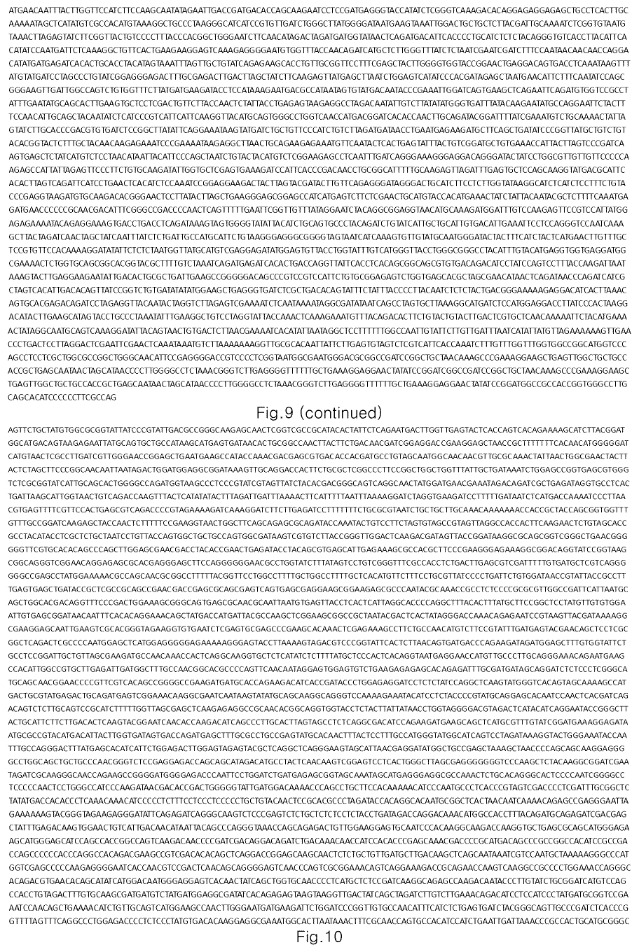

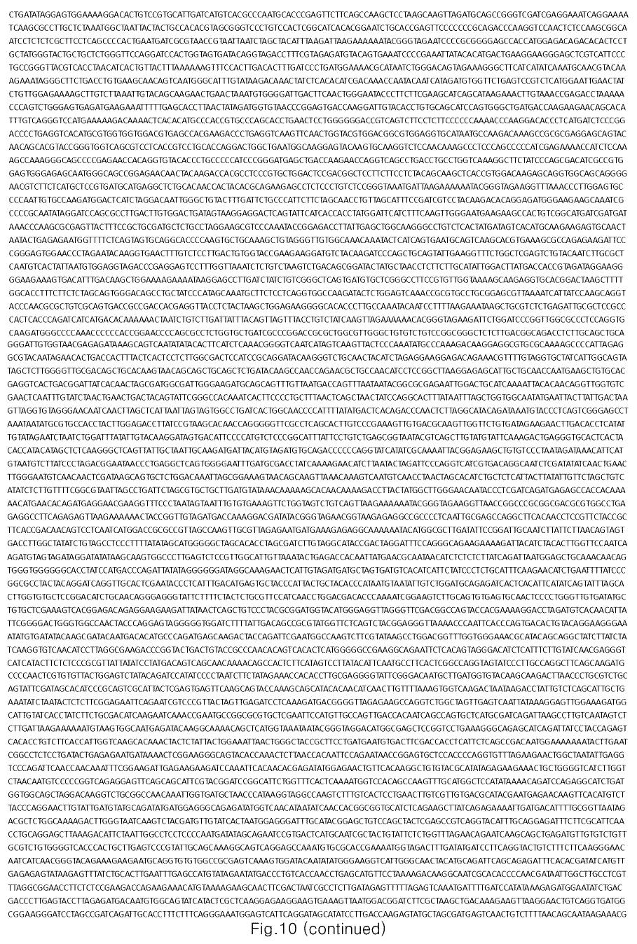

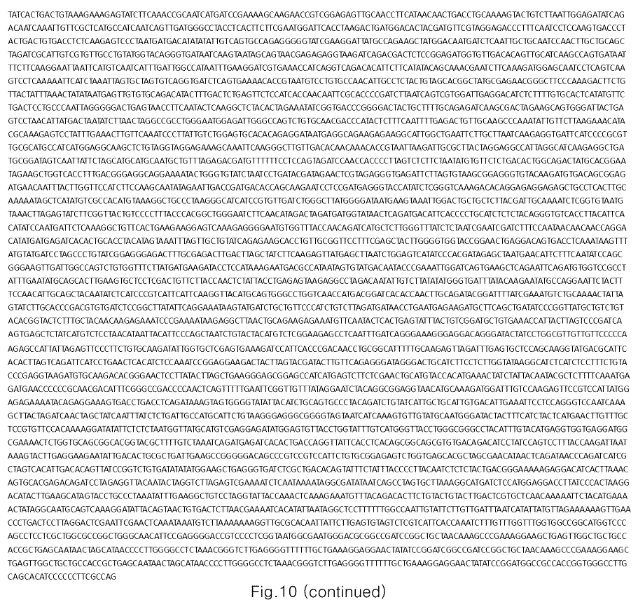

In some preferred embodiments, the sequence of the recombinant Newcastle disease virus genome is set forth in SEQ ID NO. 2 or SEQ ID NO. 5 (see FIGS. 9 and 10).

In one embodiment, the present application relates to a recombinant Newcastle disease virus, wherein the virus comprises the above-mentioned recombinant Newcastle disease virus genome.

In some preferred embodiments, a starting strain of the Newcastle disease virus can be selected from, but is not limited to: low virulent strain LaSota, Hitchner B1, or V4; medium virulent strain Mukteswar, or Anhinga; high virulent strain F48E9, JS/7/05/Ch, Italien, Herts/33, or NDV-BJ; and any chimeric strain constructed by genetic engineering means based on the starting strain, but are not limited hereto.

In one embodiment, the present application relates to a DNA molecule encoding the recombinant Newcastle disease virus genome described above (e.g., a recombinant Newcastle disease virus plasmid).

In one embodiment, the present application relates to a pharmaceutical composition, wherein the pharmaceutical composition comprises the above-mentioned recombinant Newcastle disease virus genome, recombinant Newcastle disease virus and/or DNA molecules.

In some preferred embodiments, the pharmaceutical composition further comprises a pharmaceutically acceptable excipient. The pharmaceutically acceptable excipient can be selected from, for example, but not limited to, solvent, propellant, solubilizer, cosolvent, emulsifier, colorant, disintegrant, filler, lubricant, wetting agent, osmotic pressure regulator, stabilizer, glidant, flavoring agent, preservative, suspending agent, antioxidant, penetration enhancer, pH regulator, surfactant, diluent, etc. For other available pharmaceutically acceptable pharmaceutical excipients, they can be found, for example, in “Handbook of Pharmaceutical Excipients” (4th Edition), edited by R. C. Rowe et al., translated by Junmin ZHENG, 2005, Chemical Industry Press.

In one embodiment, the present application relates to a method for preparing the above-mentioned recombinant Newcastle disease virus, wherein the method comprises:
(1) performing enzymatic cleavage on a cloning vector comprising DNA sequence of a VEGF-Trap encoding gene and a NDV viral vector, respectively, and conducting a ligation between the DNA sequence of the VEGF-Trap encoding gene and the NDV viral vector resulted from the enzymatic cleavage, so as to obtain a recombinant Newcastle disease virus plasmid;
(2) transfecting the recombinant Newcastle disease virus plasmid into cells and culturing the transfected cells to obtain the recombinant Newcastle disease virus.

In some preferred embodiments, the cloning vector can be constructed using a vector selected from PUC57 vector, pMD18-T vector, pMD19-T vector, pBlueScript SK(+/−) vector, pBluescript II KS(+/−).

In some preferred embodiments, the NDV viral vector can be a full-length cDNA sequence of the genome of a NDV virus selected from: low virulent strain LaSota, Hitchner B1, or V4; medium virulent strain Mukteswar, or Anhinga; high virulent strain F48E9, JS/7/05/Ch, Italien, Herts/33, or NDV-BJ, but are not limited hereto.

Preferably, the NDV viral vector can be pBluescript II KS(+/−)-NDV (pBrNDV), pCl-neo-NDV, or pOLTV5-NDV vector.

In the present disclosure, the recombinant Newcastle disease virus plasmid is co-transfected with helper plasmids NP, P and L (which can be any NP, P, and L recombinant plasmids obtained from constructing NP, P and L genes into any eukaryotic expression vector known in the art) capable of expressing nucleocapsid protein NP, phosphoprotein P, and RNA-dependent RNA polymerase L into the cells. In the present disclosure, the genes of helper plasmids NP, P and L can be derived from any strain of NDV, such as LaSota, Anhinga, F48E9, etc. In some preferred embodiments, the recombinant Newcastle disease virus plasmid is co-transfected into cells with helper plasmids selected from: pTM-NP, pTM-P and pTM-L; pCl-neo-NP, pCl-neo-P and pCl-neo-L; or pBluescript II KS(+/−)-NP (pBL-NP), pBluescript II KS(+/−)-P (pBL-P), and pBluescript II KS(+/−)-L (pBL-L), but not limited hereto.

Transfection herein is a technology that introduces exogenous nucleic acid substances (including DNA and RNA) into cells, mainly including threes pathways: physical mediation (electroporation, microinjection, and gene gun), chemical mediation (calcium phosphate co-precipitation, liposome transfection, cationic substance mediation), and biological mediation (protoplast transfection, virus mediated transfection). The specific operations can be conducted by the skilled in the art based on general knowledge in the art (for example, it can be seen in “Molecular Cloning: A Laboratory Manual” (4th Edition), edited by J. Sambrook et al., translated by Fuchu H E, Science Press, 2017) through selecting appropriate experimental conditions and steps, or conducted according to the instructions in commercially available kits.

In some preferred embodiments, the cells can be selected from, but are not limited to, BHK-21 cells, BSR-T7/5 cells, VERO cells, DF-1 cells, 293 cells, and MDCK cells.

The culture of the transfected cells herein can be carried out by the skilled in the art via selecting conventional culture media and culture conditions according to the type of cells (“Cell Culture (3rd Edition)”, Bin LIU, Editor in Chief, World Publishing Corporation, January 2018; “Cell Culture Technology”, Rong LAN and Zhenhui ZHOU, Editor in Chief, Chemical Industry Press, August 2007; “Tissue and Cell Culture Technology (3rd Edition)”, Jingbo ZHANG, Editor in Chief, People's Medical Publishing House, June 2014, etc.).

In one embodiment, the present application relates to use of the above-mentioned recombinant Newcastle disease virus genome, recombinant Newcastle disease virus, DNA molecule and/or pharmaceutical composition in the preparation of a medicament for treating or improving cancer.

Alternatively, the present application provides the above-mentioned recombinant Newcastle disease virus genome, recombinant Newcastle disease virus, DNA molecule and/or pharmaceutical composition for use in treating or improving cancer. Alternatively, the present application provides use of the above-mentioned recombinant Newcastle disease virus genome, recombinant Newcastle disease virus, DNA molecule and/or pharmaceutical composition for treating or improving cancer. Alternatively, a method of treating or improving cancer, comprising administering to a subject in need thereof the above-mentioned recombinant Newcastle disease virus genome, recombinant Newcastle disease virus, DNA molecule and/or pharmaceutical composition.

In some preferred embodiments, the cancer may be selected from, but is not limited to: colon cancer, liver cancer (e.g., hepatocellular carcinoma), lung cancer (e.g., non-small cell lung cancer, small-cell lung cancer), stomach cancer, rectal cancer, leukemia, lymphoma, ovarian cancer, breast cancer, endometrial cancer, bladder cancer, urothelial carcinoma, bronchogenic carcinoma, bone cancer, prostate cancer, pancreatic cancer, gallbladder cancer, bile duct cancer, esophageal cancer, renal cell carcinoma, thyroid cancer, head and neck cancer, testicular cancer, endocrine adenocarcinoma, adrenal cancer, pituitary gland cancer, skin cancer, soft tissue cancer, hemangioma, brain cancer, neurocarcinoma, eye cancer, meningioma, oropharyngeal cancer, hypopharyngeal cancer, cervical cancer, myosarcoma, uterine cancer, glioblastoma, medulloblastoma, neuroblastoma, kidney cancer, astrocytoma, glioma, meningioma, gastrinoma, neuroblastoma, melanoma, acute myeloid leukemia, myelodysplastic syndrome, or sarcoma.

Exemplary technical solutions of the present invention can be explained through the content in the following numbered paragraphs:
1. A recombinant Newcastle disease virus genome, wherein the genome comprises a VEGF-Trap encoding gene, and the VEGF-Trap encoding gene is located between P gene and M gene of the Newcastle disease virus genome.
2. The recombinant Newcastle disease virus genome of paragraph 1, wherein the VEGF-Trap encoding gene is in a form of DNA or RNA.
3. The recombinant Newcastle disease virus genome of paragraph 1 or 2, wherein the VEGF-Trap encoding gene has a sequence set forth in SEQ ID NO. 1 or a sequence having at least 80% identity thereto.
4. The recombinant Newcastle disease virus genome of any one of paragraphs 1-3, wherein the sequence of the recombinant Newcastle disease virus genome is set forth in SEQ ID NO. 2 or SEQ ID NO. 5.
5. A recombinant Newcastle disease virus, wherein the virus comprises the recombinant Newcastle disease virus genome of any one of paragraphs 1-4.
6. The recombinant Newcastle disease virus of paragraph 5, wherein a starting strain of the Newcastle disease virus is selected from: low virulent strain LaSota, Hitchner B1, or V4; medium virulent strain Mukteswar, or Anhinga; high virulent strain F48E9, JS/7/05/Ch, Italien, Herts/33, or NDV-BJ; and any chimeric strain constructed by genetic engineering means based on the starting strain.
7. A DNA molecule encoding the recombinant Newcastle disease virus genome of any one of paragraphs 1-4.
8. A pharmaceutical composition, wherein the pharmaceutical composition comprises the recombinant Newcastle disease virus genome of any one of paragraphs 1-4, the recombinant Newcastle disease virus of paragraph 5 or 6, and/or the DNA molecule of paragraph 7.
9. The recombinant Newcastle disease virus of paragraph 8, wherein the pharmaceutical composition further comprises a pharmaceutically acceptable excipient.
10. The recombinant Newcastle disease virus of paragraph 8 or 9, wherein the pharmaceutically acceptable excipient is selected from solvent, propellant, solubilizer, cosolvent, emulsifier, colorant, disintegrant, filler, lubricant, wetting agent, osmotic pressure regulator, stabilizer, glidant, flavoring agent, preservative, suspending agent, antioxidant, penetration enhancer, pH regulator, surfactant, or diluent.
11. A method for preparing the recombinant Newcastle disease virus of paragraph 5 or 6, wherein the method comprises:
(1) performing enzymatic cleavage on a cloning vector comprising DNA sequence of a VEGF-Trap encoding gene and a NDV viral vector, respectively, and conducting a ligation between the DNA sequence of the VEGF-Trap encoding gene and the NDV viral vector resulted from the enzymatic cleavage, so as to obtain a recombinant Newcastle disease virus plasmid;
(2) transfecting the recombinant Newcastle disease virus plasmid into cells and culturing the transfected cells to obtain the recombinant Newcastle disease virus.
12. The method of paragraph 11, wherein the cloning vector is constructed using a vector selected from PUC57 vector, pMD18-T vector, pMD19-T vector, pBlueScript SK(+/−) vector, pBluescript II KS(+/−).
13. The method of paragraph 11 or 12, wherein the NDV viral vector is a full-length cDNA sequence of the genome of a NDV virus selected from: low virulent strain LaSota, Hitchner B1, or V4; medium virulent strain Mukteswar, or Anhinga; high virulent strain F48E9, JS/7/05/Ch, Italien, Herts/33, or NDV-BJ.
14. The method of paragraph 13, wherein the NDV viral vector is pBluescript II KS(+/−)-NDV (pBrNDV), pCl-neo-NDV, or pOLTV5-NDV vector.
15. The method of any one of paragraphs 11-14, wherein the recombinant Newcastle disease virus plasmid is co-transfected into the cells with helper plasmids selected from: pTM-NP, pTM-P and pTM-L; pCI-neo-NP, pCl-neo-P and pCl-neo-L; or pBluescript II KS(+/−)-NP, pBluescript II KS(+/−)-P, and pBluescript II KS(+/−)-L.
16. The method of any one of paragraphs 11-15, wherein the cells are selected from BHK-21 cells, BSR-T7/5 cells, VERO cells, DF-1 cells, 293 cells, or MDCK cells.
17. Use of the recombinant Newcastle disease virus genome of any one of paragraphs 1-4, the recombinant Newcastle disease virus of paragraph 5 or 6, the DNA molecule of paragraph 7 and/or the pharmaceutical composition of any one of paragraphs 8-10 in the preparation of a medicament for treating or improving cancer.
18. The use of paragraph 17, wherein the cancer is selected from colon cancer, liver cancer, lung cancer, stomach cancer, rectal cancer, leukemia, lymphoma, ovarian cancer, breast cancer, endometrial cancer, bladder cancer, urothelial carcinoma, bronchogenic carcinoma, bone cancer, prostate cancer, pancreatic cancer, gallbladder cancer, bile duct cancer, esophageal cancer, renal cell carcinoma, thyroid cancer, head and neck cancer, testicular cancer, endocrine adenocarcinoma, adrenal cancer, pituitary gland cancer, skin cancer, soft tissue cancer, hemangioma, brain cancer, neurocarcinoma, eye cancer, meningioma, oropharyngeal cancer, hypopharyngeal cancer, cervical cancer, myosarcoma, uterine cancer, glioblastoma, medulloblastoma, neuroblastoma, kidney cancer, astrocytoma, glioma, meningioma, gastrinoma, neuroblastoma, melanoma, acute myeloid leukemia, myelodysplastic syndrome, or sarcoma.

In order to make the purpose, technical solutions and advantages of the examples of the present application clearer, the technical solutions in the examples of the present application will be clearly and completely described below. If the specific conditions are not given in the examples, they are carried out according to the conventional conditions or the conditions recommended by the manufacturer. Unless otherwise stated, the reagents, materials or instruments used are conventional products that can be purchased commercially, if the manufacturer is not indicated. The following provides a further detailed description of the features and performance of the present application in conjunction with examples.

### EXAMPLES

Unless otherwise stated, the operations of the design, synthesis and cloning of genes, as well as the construction and transfection of vectors, and electrophoresis, etc. involved in the present application can be performed according to techniques known in the art (for example, see the records in CURRENT PROTOCOLS IN MOLECULAR BIOLOGY). If not specifically specified, the technical means used in the examples are conventional means well known to the skilled in the art (for example, see “Principle and Experimental Technology of Molecular Virology”, Wei PAN, Editor in Chief, Shanghai Second Military Medical University Press, November 2002; “Fundamentals and Experimental Technologies of Medical Virology”, Zhenxiang HUANG, Editor in Chief, Science Press, February 1990, etc.).

The following examples involve the following exogenous genes: vascular inhibitor gene VEGF-Trap and human derived angiostatin gene Angiostatin (Genbank accession no. NG_016200.1).

pMD19-T was purchased from TaKaRa Bioengineering (Dalian) Co., Ltd. (Dalian TaKaRa Company). BHK-21 cell (baby hamster kidney cell), human colon cancer cell HCT116, mouse colon cancer cell CT26, mouse breast cancer cell 4T1, and human umbilical vein endothelial cell EA.hy926 were all purchased from ATCC.

DMEM (high glucose) medium, McCoy'5A medium, RPMI 1640 medium, trypsin, newborn calf serum (FCS), and fetal bovine serum (FBS) were purchased from GIBCO company. SPF chicken embryos were purchased from Beijing Boehringer Ingelheim Viton Biotechnology Co., Ltd. Balb/c mice (Kunming mice) were purchased from Sipeifu (Beijing) Biotechnology Co., Ltd.

### Example 1 Preparation of Recombinant Newcastle Disease Virus

1. Construction of a Recombinant Newcastle Disease Virus Plasmid Inserted with Exogenous Genes (Taking pBrNDV-VEGF-Trap for Example)

According to the records in the literature of Jocelyn Holash et al. (VEGF-Trap: A VEGF blocker with potent antitumor effects, August 2002; https://doi.org/10.1073/pnas.172398299), the sequence of the VEGF-Trap gene (SEQ ID NO. 1) was obtained which was shown as follows:

TCCCCGCGGGGAGCCACCATGGAGACAGACACACTCCTGCTATGGGTAC
TGCTGCTCTGGGTTCCAGGATCCACTGGTAGTGATACAGGTAGACCTTT
CGTAGAGATGTACAGTGAAATCCCCGAAATTATACACATGACTGAAGGA
AGGGAGCTCGTCATTCCCTGCCGGGTTACGTCACCTAACATCACTGTTA
CTTTAAAAAAGTTTCCACTTGACACTTTGATCCCTGATGGAAAACGCAT
AATCTGGGACAGTAGAAAGGGCTTCATCATATCAAATGCAACGTACAAA
GAAATAGGGCTTCTGACCTGTGAAGCAACAGTCAATGGGCATTTGTATA
AGACAAACTATCTCACACATCGACAAACCAATACAATCATAGATGTGGT
TCTGAGTCCGTCTCATGGAATTGAACTATCTGTTGGAGAAAAGCTTGTC
TTAAATTGTACAGCAAGAACTGAACTAAATGTGGGGATTGACTTCAACT
GGGAATACCCTTCTTCGAAGCATCAGCATAAGAAACTTGTAAACCGAGA
CCTAAAAACCCAGTCTGGGAGTGAGATGAAGAAATTTTTGAGCACCTTA
ACTATAGATGGTGTAACCCGGAGTGACCAAGGATTGTACACCTGTGCAG
CATCCAGTGGGCTGATGACCAAGAAGAACAGCACATTTGTCAGGGTCCA
TGAAAAAGACAAAACTCACACATGCCCACCGTGCCCAGCACCTGAACTC
CTGGGGGGACCGTCAGTCTTCCTCTTCCCCCCAAAACCCAAGGACACCC
TCATGATCTCCCGGACCCCTGAGGTCACATGCGTGGTGGTGGACGTGAG
CCACGAAGACCCTGAGGTCAAGTTCAACTGGTACGTGGACGGCGTGGAG
GTGCATAATGCCAAGACAAAGCCGCGCGAGGAGCAGTACAACAGCACGT
ACCGGGTGGTCAGCGTCCTCACCGTCCTGCACCAGGACTGGCTGAATGG
CAAGGAGTACAAGTGCAAGGTCTCCAACAAAGCCCTCCCAGCCCCCATC
GAGAAAACCATCTCCAAAGCCAAAGGGCAGCCCCGAGAACCACAGGTGT
ACACCCTGCCCCCATCCCGGGATGAGCTGACCAAGAACCAGGTCAGCCT
GACCTGCCTGGTCAAAGGCTTCTATCCCAGCGACATCGCCGTGGAGTGG
GAGAGCAATGGGCAGCCGGAGAACAACTACAAGACCACGCCTCCCGTGC
TGGACTCCGACGGCTCCTTCTTCCTCTACAGCAAGCTCACCGTGGACAA
GAGCAGGTGGCAGCAGGGGAACGTCTTCTCATGCTCCGTGATGCATGAG
GCTCTGCACAACCACTACACGCAGAAGAGCCTCTCCCTGTCTCCGGGTA
AATGATTAAGAAAAAATACGGGTAGAAGGTTTAAACCC

The recombinant plasmid pBrNDV-VEGF-Trap was constructed according to the following method:
1. The VEGF-Trap gene containing the enzymatic cleavage sites of SacII enzyme (5′) and PmeI enzyme (3′) was synthesized and ligated to the pUC57 vector to form pUC57-VEGF-Trap by Sangon Biotech (Shanghai) Co., Ltd., which was used for subsequent experiments.
2. The plasmid pUC57-VEGF-Trap in step 1 was cleaved by the restriction endonucleases PmeI and SacII (purchased from NEB Company) according to the instructions of the manufacturer. The enzymatically cleaved product was identified by nucleic acid agarose gel electrophoresis. After it was identified as correct, the enzymatically cleaved product was recovered by the gel purification kit (purchased from Tiangen Biotech (Beijing) Co., Ltd., Cat. No.: DP219) according to the instructions of the manufacturer.
3. The plasmid pBrNDV (purchased from NEB) was cleaved by the restriction endonucleases PmeI and SacII according to the instructions of the manufacturer, and the plasmid vector was recovered by the gel purification kit (purchased from Tiangen Biotech (Beijing) Co., Ltd., Cat. No.: DP219) according to the instructions of the manufacturer.
4. The enzymatically cleaved product in step 2 was ligated with the vector in step 3 by using T4 DNA ligase (purchased from NEB) according to the instructions of the manufacturer to obtain the recombinant Newcastle disease virus plasmid pBrNDV-VEGF-Trap, wherein the VEGF-Trap gene was inserted between P gene and M gene of the plasmid. The PCR was performed using Takara Bio taq enzyme (purchased from Takara Bio) according to the instructions of the manufacturer with the above recombinant plasmid (upstream primer: 5′ TCAAGCGCCTTGCTCTAAATGGC 3′ (SEQ ID NO. 3); downstream primer: 5′ GGGCAGAATCAAAGTACAGCCCAAT 3′ (SEQ ID NO. 4)), and the double enzyme digestion identification was performed with PmeI and Sacll (37° C., 1h); the correct plasmid samples were packaged and sent to Sangon Biotech (Shanghai) Co., Ltd. for sequencing. The sequencing results were aligned using the sequence analysis software DNAMAN. After sequencing, the sequenced sequence was consistent with the target sequence.

The following recombinant plasmid pBrNDV-Angiostatin inserted with the exogenous Angiostatin gene (NG_016200.1) was constructed and identified using the same method as above (primers for PCR amplification were the same as above).

II. Preparation of Recombinant Newcastle Disease Virus

The recombinant Newcastle disease virus rNDV-VEGF-Trap was prepared using the above recombinant Newcastle disease virus plasmid by the following method:
1. Using liposome transfection technology, the successfully constructed recombinant Newcastle disease virus plasmid pBrNDV-VEGF-Trap was co-transfected with three helper plasmids pBL-NP, pBL-P and pBL-L (constructed according to the method described in Jinying G E, Basic and applied research on reverse genetic operation of Newcastle disease virus, 2006) into a monolayer BHK-21 cell stably expressing T7 RNA polymerase. The operation steps were performed according to the instructions of Lipofectamine 3000 transfection reagent (purchased from Invitrogen). After 72 hours of incubation, it was repeatedly frozen and thawed 3 times at −80° C., centrifuged under 12000 rpm at 4° C. to collect the supernatant, into which 0.001% trypsin was added.
2. 200 μL of the supernatant obtained in step 1 was taken, inoculated into the allantoic cavity of 9-day old SPF grade chicken embryos, and then cultured in an incubator at 37° C., 5% CO₂ for 72 hours to obtain the allantoic fluid. Hemagglutination titer test was performed (for example, the method described in Ling ZHOU, Yanfang L I, Xiali M A, Isolation and Identification of a chicken-derived Newcastle disease virus [J], Zhejiang Animal Husbandry and Veterinary Medicine, 2015, 40(03):8-10). The positive allantoic fluid was frozen and stored in a refrigerator at −80° C., and the recombinant Newcastle disease virus successfully rescued was named as rNDV-VEGF-Trap.
3. The recombinant viral RNA was extracted from the recombinant Newcastle disease virus obtained in step 2 according to the instructions of QIAamp Viral RNA Mini Kit(50), and cDNA samples were obtained by random primers. The RT-PCR amplification (Thermo Fisher RT-PCR kit, the first strand of cDNA was synthesized and PCR was performed according to the instructions of the kit) was performed for the inserted VEGF-Trap gene using P/M site primers P/M-F (the sequence thereof was the same as SEQ ID NO. 3) and P/M-R the sequence thereof was the same as SEQ ID NO. 4). The amplified PCR products were sent to Sangon Biotech (Shanghai) Co., Ltd. for sequencing. The sequencing results were aligned using the sequence analysis software DNAMAN. After sequencing, the sequenced sequence was consistent with the target sequence.
4. The virus with the exogenous gene being sequenced as correct in step 3 was taken, inoculated into the allantoic cavity of new 9 to 11-day old SPF grade chicken embryos, and cultured at 37° C. for 72 h. The chicken embryo allantoic fluid was collected for HA detection (for example, performed by referring to the relevant methods recorded in Ling Z H O U, Yanfang L I, Xiali M A, Isolation and Identification of a chicken-derived Newcastle disease virus [J], Zhejiang Animal Husbandry and Veterinary Medicine, 2015, 40(03):8-10). The allantoic fluid with a HA titer greater than 29 was selected for mixing and then packaged for use.
5. The allantoic fluid of each group was collected and used for Western Blot (for example, it could be referred to the relevant method recorded in An Y et al., Recombinant Newcastle disease virus expressing P53 demonstrates promising antitumor efficiency in hepatoma model [J]. Journal of Biomedical Science, 2016 23(1): 55) to detect the expression of each exogenous gene. The results showed that the recombinant Newcastle disease virus rNDV-VEGF-Trap could stably express the exogenous gene VEGF-Trap (see FIG. 1).

Likewise, the recombinant Newcastle disease virus rNDV-Angiostatin was prepared according to the above method.

In the following description, unless otherwise specified, rNDV-VEGF-Trap and rNDV-Angiostatin refer to the recombinant viruses prepared by the above method using recombinant plasmids pBrNDV-VEGF-Trap and pBrNDV-Angiostatin, respectively.

The parental virus in the following Examples 2-3 refers to the virus obtained via the following modification: starting from the Newcastle disease virus LaSota (purchased from Harbin Veterinary Epidemic Prevention Station), replacing F gene of the LaSota strain with F gene of the high virulent strain F48E9 of Newcastle disease virus (GenBank accession no.: AY508514.1) according to the engineering method of “gene replacement” described in Yong WANG et al. (Evaluation of Newcastle disease virus with derivated Hemagglutinin-Neuraminidase gene of mesogenic strain, Acta Microbiologica Sinica, 2008, 48(5): 638-643). The parental strain was named as rNDV herein.

### Example 2 Detection of Proliferation Stability of Recombinant Virus

Detection of TCID50 was performed according to the following method:
1. 10,000 DF-1 cells were inoculated with DMEM medium supplemented with 10% FBS and 1% antibiotics into a 96-well microplate, and cultured overnight in a 37° C., 5% CO₂ incubator.
2. Before inoculating each recombinant Newcastle disease virus (rNDV-VEGF-Trap and rNDV-Angiostatin) and the parental virus, the original cell culture medium in the plate of step 1 was discarded, into which 180 μL fresh DMEM culture medium supplemented with 10% allantoic fluid and 1% antibiotics was added.
3. 20 μL of each recombinant Newcastle disease virus and parental virus were inoculated into the top row of wells, respectively. After being mixed by pipetting, 20 μL of each mixed solution was pipetted into the lower wells for continuous 10-fold gradient dilution. Each virus was set up in triplicate.
4. After the inoculated recombinant Newcastle disease virus and parental virus were incubated for 1 hour in a 37° C., 5% CO₂ incubator, the culture medium was discarded, and it was washed once with 0.2 mL 1×PBS buffer, into which 200 μL fresh DMEM culture medium supplemented with 10% allantoic fluid and 1% antibiotics was added.
5. The culture was continued in a 37° C., 5% CO₂ incubator, and the cell lesion holes were observed under an optical inverted microscope after 12 h, 24 h, 36 h, 48 h, 60 h, and 72 h and the number of the lesion holes was recorded, respectively. The titer of the virus was calculated using the Reed and Muench method, and a proliferation curve was drawn.

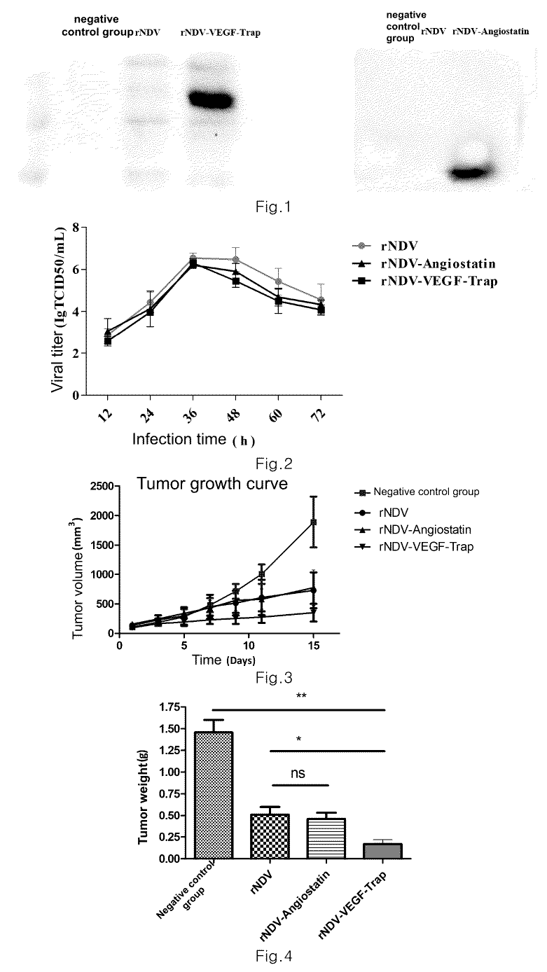

The results were shown in FIG. 2. The proliferation trends of the recombinant Newcastle disease viruses rNDV-VEGF-Trap and rNDV-Angiostatin were not significantly different from that of the parental virus, indicating that the insertion of exogenous genes did not affect virus proliferation.

### Example 3 Therapeutic Effect of Recombinant Newcastle Disease Virus on Tumors

1 Establishment of Balb/c Mouse Colon Cancer Tumor-Bearing Animal Model

Mouse colon cancer cell CT26 was taken and stained with trypan blue to determine that the cell viability was 95% or more, which was diluted with physiological saline to a cell suspension of 1×10⁶/mL, and the cell suspension was injected subcutaneously into the right abdomen at a dose of 0.1 mL per Balb/c mouse. After 8-12 days, the diameter of the solid tumor reaches 5-8 mm, indicating that the model was successfully constructed and subsequent experiments could be carried out. Individuals with large differences in tumor shape and size were excluded, and mice with tumor diameters of 5-8 mm were selected as model mice.

2 The Therapeutic Effect of Recombinant Viruses on Tumors

The model mice were randomly divided into 4 groups, 10 mice per group, and were treated as follows:

rNDV-VEGF-Trap group: 0.2 mL of the PBS suspension of the rNDV-VEGF-Trap virus prepared in Example 1 (prepared with 1×PBS buffer; containing 10⁷ pfu virus) was injected into the tumor of model mice every day for 14 days;

rNDV-Angiostatin group: 0.2 mL of the PBS suspension of the rNDV-Angiostatin virus prepared in Example 1 (prepared with 1×PBS buffer; containing 10⁷ pfu virus) was injected into the tumor of model mice every day for 14 days;

rNDV group: 0.2 mL of the PBS suspension of the parental virus (prepared with 1×PBS buffer; containing 10⁷ pfu virus) was injected into the tumor of model mice every day for 14 days;

Negative control group (model group): 0.2 mL of SPF chick embryo allantoic fluid was injected into the tumor of model mice every day for 14 days.

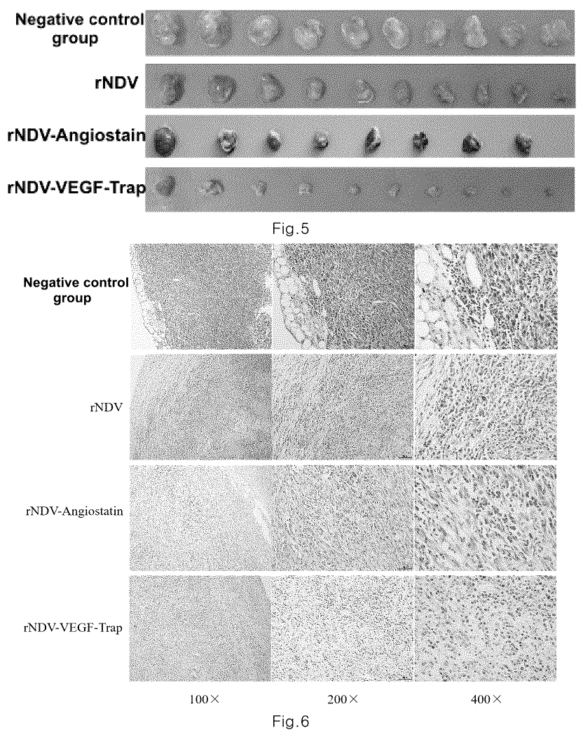

From the day of treatment, tumor volume was measured every other day, and a tumor growth curve was made based on the measurement results (FIG. 3). After the treatment, mice were euthanized, and tumors were removed, and tumor weight and size were measured (results thereof were shown in FIG. 4 and FIG. 5). The average tumor volume of the negative control group was 1889.17 mm³, the average tumor volume of the parental NDV (rNDV) treatment group was 728.49 mm³, the average tumor volume of the rNDV-Angiostatin treatment group was 774.37 mm³; the average tumor volume of the rNDV-VEGF-Trap treatment group was 350.36 mm³. The results showed that compared with the negative control group, both the parental virus and the recombinant Newcastle disease virus had a significant inhibitory effect on tumor growth, wherein the anti-tumor effect of rNDV-Angiostatin was not significantly different from that of the rNDV group, while the anti-tumor effect of rNDV-VEGF-Trap was significantly higher than that of the rNDV group.

3 Observation of Tumor Pathological Sections

To observe the inhibitory effect of recombinant Newcastle disease virus rNDV-VEGF-Trap on colon cancer and related blood vessels, tumor tissues from each group of mice were taken, and fixed with 4% paraformaldehyde. Paraffin sections of the tumor tissues were prepared with a thickness of 4 μm as follows. The tumor tissue morphology of each group was observed under a microscope after HE staining, and the expression of CD34 protein in the tumor tissue of each group was detected by immunohistochemical staining. The specific operations were as follows:

3.1 Preparation of Paraffin Sections
(1) The paraffin was put into a 1 L beaker, into which beeswax was added, and it was put into a wax box at 60° C. When the wax was completely melted, filtered with filter paper, taken out and cooled at room temperature, and then put back into the wax box to melt, repeating 2-3 times.
(2) The slides were placed one by one into the prepared washing solution (concentrated potassium dichromate solution: 25 g potassium dichromate, 75 mL water, 400 mL concentrated sulfuric acid) and immersed for 24 h, rinsed thoroughly with tap water, and further immersed in 95 vol % alcohol for 24 h, and then the slides were dried with lens paper and sterilized by dry heat at 180° C. for 6 h. After sterilization, the slides were immersed one by one into a dye vat containing APES treatment agent (APES: acetone=1:50) for 5 min. After being taken out, they were rinsed twice with distilled water and placed in a slice box, and dried at 60° C. for later use.
(3) After the above treatment, the lung tissues of mice in each group were taken and fixed in a pre-prepared 4% neutral formaldehyde fixative solution for 48 h. The tissues were taken out and the tissue blocks were trimmed to a size of about 1 cm with a blade.
(4) Dehydration and transparency: 30 vol % ethanol-30 min, 50 vol % ethanol-30 min, 70 vol % ethanol overnight at 4° C.; the next day, 80 vol % ethanol-30 min, 90 vol % ethanol-30 min, 100 vol % ethanol-30 min (2 times). In a fume hood, the dehydrated tissue was immersed in a dye vat with xylene:absolute ethanol=1:1 and rinsed for 20 min, and then rinsed in pure xylene for 20 min, repeating twice. At this time, the color of the tissue block becomes deeper and transparent, and the xylene solution becomes clear.
(5) Wax dipping: the transparent tissue was transferred into the fully melted paraffin in (1), and placed in an incubator at 60° C. for 120 min.
(6) Embedding: the paraffin in the wax box was poured into a paper box, small tweezers were used to arrange the tissue blocks in order, ensuring that the cutting surface was facing down.
(7) Paraffin block trimming: after the paraffin in the paper box being solidified, the paraffin block was taken out and trimmed into a trapezoid using a blade. The distance between the edge of the tissue and the edge of the paraffin block should not be less than 2 mm.
(8) Sectioning: sectioning was continuously carried out with a thickness of about 4 μm. The sections were unfolded in the water bath of the Water Bath-Slide Drier at about 40° C. The slides treated in (2) were used to take out the sections from the water bath and place them on the frame of the Water Bath-Slide Drier. After being slightly dried, they were immediately placed in an oven at 37° C. overnight to make the sections close to the slide and translucent. After staying overnight, the slides were placed in the slide box and ready for use.

3.2 HE Staining
(1) Dewaxing: when the wax box was preheated to a temperature of 60° C., the prepared tumor tissue sections were put into an oven at 60° C. for 1 h, immersed in xylene for 5 min (3 times) after the paraffin on the sections being melted. Absolute ethanol-2 min; 90 vol % ethanol-5 min; 80 vol % ethanol-5 min; 70 vol % ethanol-5 min; 50 vol % ethanol-5 min; water-5 min (3 times).
(2) Staining: the sections were stained with hematoxylin for 30 sec and then rinsed with distilled water for 1 min; they were inserted into an eosin dye vat and taken out immediately, and immersed in distilled water for 1 min (3 times).
(3) Dehydration: 50 vol % ethanol-1 min; 70 vol % ethanol-1 min; 80 vol % ethanol-1 min; 90 vol % ethanol-1 min; 100 vol % ethanol-5 min (2 times); xylene 1-5 min (2 times).
(4) Neutral gum sealing: the cover glass was placed flat after being dried, to the center of which 1-2 drops of neutral gum were added, and the tissue side of the slide was gently covered on the cover glass, allowing the neutral gum to fully spread along the cover glass; then the slide was tilted and the filter paper was used to absorb excess xylene while taking care to avoid the generation of air bubbles. The slides were dried and stored in a slide box, and the pathological changes of tumor tissues in each group of the mice were observed and compared under a microscope.

3.3 Immunohistochemical Staining
1. Dewaxing and hydration: the prepared paraffin sections were immersed into xylene twice for dewaxing, 5 min each time. Then, they were placed into various levels of alcohol solutions of 100 vol %, 95 vol %, 90 vol %, 80 vol %, and 70 vol % for 5 min each, and then rinsed in distilled water twice, 3 min each time.
2. Antigen retrieval: 0.01 M sodium citrate buffer (pH=6.0) was heated in water bath to 95° C., and then the slices were placed therein and heated for 10 min. They were rinsed 3 times with 1×PBS buffer, 5 min each time.
3. Inactivation of endogenous peroxidase: an appropriate amount of endogenous peroxidase blocking buffer (purchased from Beyotime; Cat. No. P0100A) was added dropwise to completely cover the sample, and they were incubated at room temperature for 10 min. They were washed 3 times with 1×PBS buffer, 3 min each time.
4. Blocking: a blocking buffer (purchased from Beyotime; Cat. No. P0260 QuickBlock) was added dropwise to block the tissue sections for 10 min.
5. Incubation of the primary antibody: the blocking buffer was used to prepare a CD34 primary antibody working solution (purchased from Abcam) according to the dilution ratio in the instructions of the antibody. The primary antibody working solution was added dropwise to each tissue section and incubated at 4° C. overnight. After incubation with the primary antibody (anti-CD34 antibody, purchased from Abcam), the tissue sections were washed 3 times with 1×PBST buffer, 5 min each time.
6. Incubation of the secondary antibody: the blocking buffer was used to prepare a goat anti-rabbit secondary antibody (purchased from Abcam) working solution according to the dilution ratio in the instructions of the antibody. The secondary antibody working solution was added dropwise to each tissue section and incubated at room temperature for 1 h. After incubation with the secondary antibody, the tissue sections were washed 3 times with 1×PBST buffer, 5 min each time.
7. Color development: 100 μL of DAB color development working solution (purchased from Beyotime) was added dropwise to fully cover the sample. The incubation was performed at room temperature in dark for 15 min. After color development, the DAB color development working solution was removed, and it was washed 1-2 times with distilled water to terminate the color development reaction.

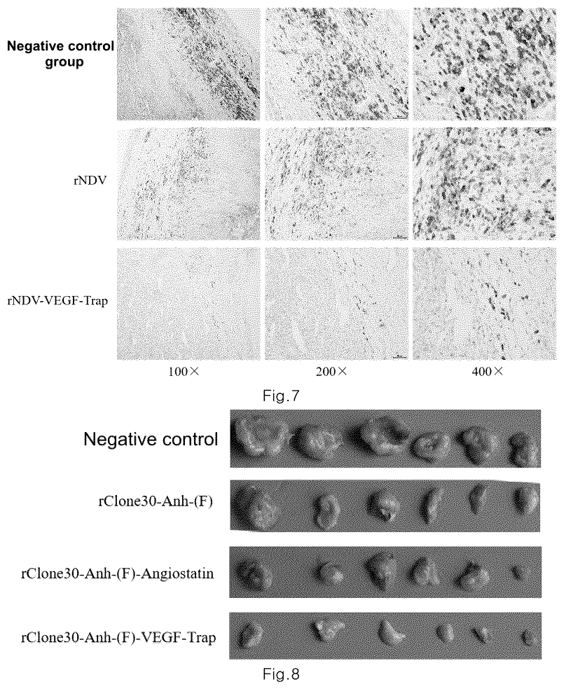

The results showed that after the treatment, the tumor tissue structure of the mice in the negative control group (model group) was dense, with intact cell morphology and vigorous growth; the tumor lesions of the mice in the rNDV group disintegrated and the tumor cell structure was relatively loose; the tumor structure of the mice in rNDV-Angiostatin group was not significantly different from that of the rNDV group, while the tumor tissue lesions of the mice in rNDV-VEGF-Trap group were extensively disintegrated, and the tumor cell structure was very loose, immune cells infiltrated in many places, and the tumor cells were scattered individually (see FIG. 6). Immunohistochemical staining results showed that the expression of CD34 in the mice of the model group was abundant, the rNDV group was similar to the model group, while the expression of CD34 in the mice of the rNDV-VEGF-Trap group was significantly reduced. It showed that rNDV-VEGF-Trap had the therapeutic effect on inhibiting the proliferation of vascular endothelial cells (see FIG. 7).

### Example 4 Safety Testing of the Recombinant Newcastle Disease Virus

Healthy 6-week old SPF grade Balb/c mice were selected and grouped, 10 mice per group. The mice in the control group were fed normally. Each mouse in the test group was intraperitoneally injected with 5×10⁸ pfu (10 times the therapeutic dose) of the recombinant Newcastle disease virus rNDV-VEGF-Trap and then observed for 30 days. Mice with obvious adverse reactions such as listlessness, bristled fur and death were considered positive.

The results showed that on the second day of injection, the fur of 3 mice in the test group stood up, and their diet and water intakes were not affected. After one week of continuous injection of the recombinant Newcastle disease virus rNDV-VEGF-Trap, the fur of the mice in the test group returned to normal. After continued observation for one month, no mice in the test group showed any obvious adverse reactions (including listlessness and bristled fur), and no mice died.

Therefore, the recombinant Newcastle disease virus rNDV-VEGF-Trap prepared in the present application had good safety.

### Example 5

The oncolytic virus rClone30-Anh-(F) as the parental strain provided in the following example was obtained via the following modification: starting from the Newcastle disease virus LaSota strain (purchased from Harbin Veterinary Epidemic Prevention Station), replacing F gene of the LaSota strain with F gene of the medium virulent strain Anhinga of Newcastle disease virus (GenBank accession no.: EF065682.1) according to the engineering method of “gene replacement” described in Yong WANG eta. (Acta Microbiologica Sinica, 2008, 48(5): 638-643, supra). The genome of the recombinant Newcastle disease virus expressing VEGF-Trap in this example was set forth in SEQ ID NO. 5 (see FIG. 10); the nucleic acid sequence of VEGF-Trap was set forth in SEQ ID NO. 1.

First, according to the method described in Example 1, the VEGF-Trap gene (SEQ ID NO. 1) and the Angiostatin gene (NG_016200.1) were used to construct the corresponding recombinant Newcastle disease virus plasmids and the recombinant Newcastle disease viruses respectively, and the recombinant Newcastle disease viruses were successfully rescued. The successfully rescued and identified the correct recombinant Newcastle disease viruses were named as rClone30-Anh-(F)-VEGF-Trap and rClone30-Anh-(F)-Angiostatin, respectively.

Then, H22 subcutaneous tumor-bearing model (i.e., mouse liver cancer model) was established using H22 cells (purchased from Nanjing CoBioer) according to the method described in Example 4. When the tumor grew to about 100 mm³, 100 μL of PBS suspensions (prepared using 1×PBS buffer) of each of the above-mentioned oncolytic viruses (rClone30-Anh-(F)-Angiostatin and rClone30-Anh-(F)-VEGF-Trap) and the parental strain rClone30-Anh-(F) were injected into the tumor. In each treatment group, each oncolytic virus (rClone30-Anh-(F)-Angiostatin, rClone30-Anh-(F)-VEGF-Trap and rClone30-Anh-(F)) was injected into the tumor once a day for a total of 14 days, with 1×10⁷ PFU injected each time; 1×PBS buffer (without oncolytic virus) was injected into the tumor of the mouse liver cancer model as a negative control group (also named as “PBS treatment group”); 6 animals per group; tumor tissues were dissected to observe the therapeutic effects of each recombinant virus.

As shown in FIG. 8, after the treatment, the average tumor volume of the negative control group was 1421.77 mm³, the average tumor volume of the parental rClone30-Anh-(F) treatment group was 807.30 mm³, the average tumor volume of the rClone30-Anh-(F)-Angiostatin treatment group was 668.60 mm³, and the average tumor volume of the rClone30-Anh-(F)-VEGF-Trap treatment group was 326.05 mm³. The results showed that compared with the negative control group, the parental strain rClone30-Anh-(F) treatment group, rClone30-Anh-(F)-Angiostatin treatment group and rClone30-Anh-(F)-VEGF-Trap treatment group all could inhibit tumor growth, and especially the rClone30-Anh-(F)-VEGF-Trap treatment group had the smallest average tumor volume.

The above descriptions are only preferred Examples of the present invention and are not intended to limit the present invention. For the skilled in the art, the present invention may have various modifications and changes. Any modifications, equivalent substitutions, improvements, etc. made within the spirit and principle of the present invention shall be included in the protection scope of the present invention.

For the purpose of description and disclosure, all patents, patent applications, and other publications are expressly incorporated herein by reference. These publications are provided solely for their disclosures prior to the filing date of the present application. All statements regarding the dates of these documents or the representation of the contents of these documents are based on the information available to the applicant, and do not constitute any admission as to the correctness of the dates of these documents or the contents of these documents. Besides, in any country, any reference to these publications herein does not constitute an admission that the publications form part of the common knowledge in the art.

1. A recombinant Newcastle disease virus genome, wherein the genome comprises a VEGF-Trap encoding gene, and, wherein the VEGF-Trap encoding gene is located between P gene and M gene of the Newcastle disease virus genome.

2-15. (canceled)

16. The recombinant Newcastle disease virus genome of claim 1, wherein the VEGF-Trap encoding gene is in a form of DNA or RNA.

17. The recombinant Newcastle disease virus genome of claim 1, wherein the VEGF-Trap encoding gene has the sequence set forth in SEQ ID NO. 1 or a sequence having at least 80% identity thereto.

18. The recombinant Newcastle disease virus genome of claim 1, wherein the sequence of the recombinant Newcastle disease virus genome is set forth in SEQ ID NO. 2 or SEQ ID NO. 5.

19. A recombinant Newcastle disease virus, wherein the virus comprises the recombinant Newcastle disease virus genome of claim 1.

20. The recombinant Newcastle disease virus of claim 19, wherein a starting strain of the Newcastle disease virus is selected from a low virulent strain LaSota, Hitchner B1, or V4; a medium virulent strain Mukteswar, or Anhinga; a high virulent strain F48E9, JS/7/05/Ch, Italien, Herts/33, or NDV-BJ; or any chimeric strain based on the starting strain.

21. A pharmaceutical composition, wherein the pharmaceutical composition comprises the recombinant Newcastle disease virus genome of claim 1.

22. The pharmaceutical composition of claim 21, wherein the pharmaceutical composition further comprises a pharmaceutically acceptable excipient.

23. The pharmaceutical composition of claim 21, wherein the recombinant Newcastle disease virus genome is comprised in a recombinant Newcastle disease virus.

24. The pharmaceutical composition of claim 21, wherein the VEGF-Trap encoding gene is in a form of DNA or RNA.

25. The pharmaceutical composition of claim 24, wherein the VEGF-Trap encoding gene has the sequence set forth in SEQ ID NO. 1 or a sequence having at least 80% identity thereto.

26. The pharmaceutical composition of claim 21, wherein the sequence of the recombinant Newcastle disease virus genome is set forth in SEQ ID NO. 2 or SEQ ID NO. 5.

27. The pharmaceutical composition of claim 22, wherein the pharmaceutically acceptable excipient is a solvent, propellant, solubilizer, cosolvent, emulsifier, colorant, disintegrant, filler, lubricant, wetting agent, osmotic pressure regulator, stabilizer, glidant, flavoring agent, preservative, suspending agent, antioxidant, penetration enhancer, pH regulator, surfactant, or diluent.

28. A method of treating or inhibiting a cancer, comprising administering to a subject in need thereof the recombinant Newcastle disease virus genome of claim 1.

29. The method of claim 28, wherein the recombinant Newcastle disease virus genome is comprised in a recombinant Newcastle disease virus.

30. The method of claim 28, wherein the cancer is selected from the group consisting of colon cancer, liver cancer, lung cancer, stomach cancer, rectal cancer, leukemia, lymphoma, ovarian cancer, breast cancer, endometrial cancer, bladder cancer, urothelial carcinoma, bronchogenic carcinoma, bone cancer, prostate cancer, pancreatic cancer, gallbladder cancer, bile duct cancer, esophageal cancer, renal cell carcinoma, thyroid cancer, head and neck cancer, testicular cancer, endocrine adenocarcinoma, adrenal cancer, pituitary gland cancer, skin cancer, soft tissue cancer, hemangioma, brain cancer, neurocarcinoma, eye cancer, meningioma, oropharyngeal cancer, hypopharyngeal cancer, cervical cancer, myosarcoma, uterine cancer, glioblastoma, medulloblastoma, neuroblastoma, kidney cancer, astrocytoma, glioma, meningioma, gastrinoma, neuroblastoma, melanoma, acute myeloid leukemia, myelodysplastic syndrome, and sarcoma.
